(19) United States
(12) Patent Application Publication
(10) Pub. No.: US 2018/0117159 A1
(43) Pub. Date: May 3, 2018

(54) Method for producing solid formulations with improved dissolution by annealing with an adjuvant

(71) Applicant: Robert Arthur Bellantone, Yonkers, NY (US)

(72) Inventor: Robert Arthur Bellantone, Yonkers, NY (US)

(21) Appl. No.: 15/732,352

(22) Filed: Oct. 30, 2017

Related U.S. Application Data
(60) Provisional application No. 62/496,670, filed on Oct. 25, 2016.

(51) Int. Cl. A61K 47/32; A61K 31/405

(52) U.S. Cl. CPC A61K 47/32; A61K 31/405

(57) ABSTRACT

The invention provides a method for producing mixtures of a drug and polymer that provide improved dissolution (faster or more complete dissolution) compared to pure drug or drug and polymer physical mixtures. The method uses annealing of a mixture of drug and polymer in contact with an adjuvant, which allows improved dissolution to be achieved with shorter annealing times and/or reduced annealing temperatures compared to annealing without an adjuvant, without dissolving the drug and polymer in a solvent and without melting the polymer.

### CROSS-REFERENCE TO RELATED APPLICATION

This invention claims priority of U.S. provisional patent application No. 62/496,670, filed Oct. 25, 2016.

### BACKGROUND OF THE INVENTION

### Field of the Invention

The invention relates to an improved method of making solid formulations of drugs with poor aqueous solubility mixed with polymers, such that they show improved dissolution compared to the pure drug or physical mixtures of the drug and polymer. In particular, the invention relates to a technique that employs an adjuvant to allows producing said formulations by annealing at lower temperatures and for shorter times than required by techniques such as melt-mix, and without dissolving the drug or polymer. The method is referred to as annealing with an adjuvant,

Examples include pharmaceutical oral dosage forms, where improved dissolution of drugs with poor aqueous solubility in GI fluids can increase absorption into the bloodstream. However, the instant invention is not limited to pharmaceutical dosage forms.

### Summary of the Prior Art

An important problem in pharmaceutics is to improve the dissolution of drugs with poor aqueous solubility compared to the dissolution of the pure drug, or a physical mixture of the drug and a polymer. In this context, improving dissolution refers to increasing the dissolution rate, the extent of dissolution (how much of the drug is dissolved), or both. Improved dissolution can be demonstrated by in vitro dissolution experiments using techniques known in the art, and it can be implied in vivo. For instance, for drugs with poor solubility in water that are administered orally, improving the dissolution can improve the bioavailability of the drug, meaning the fraction of the given drug that is absorbed into the bloodstream and reaches the systemic circulation is increased compared to slower dissolution.

Improving the dissolution of drugs with poor aqueous solubility can be accomplished by techniques known in the art, including producing salts, reducing particle size of pure drug crystals, solubilizing the drug by incorporation into micelles and complexation with polymers and cyclodextrins, etc. However, one of the most important techniques is by formulating the drug as a solid solution or solid dispersion in a carrier such as a polymer.

In this context, dissolution refers to the rate at which drug molecules leave the undissolved state and molecularly disperse in a solvent, either as individual molecules but perhaps also as complexed to a water-soluble polymer. In the undissolved state, the drug is typically but not necessarily in a solid form. In principle, dissolution can occur in any medium, but in this context, dissolution into water or other aqueous liquid medium such as a buffer or surfactant solution is most relevant to pharmaceutical applications.

Dissolution is a process and should be distinguished from solubility. The term solubility (sometimes referred to as the equilibrium solubility, or thermodynamic solubility) is known in the art and refers to the maximum concentration of a drug or other solute that can be dissolved in a solvent with no tendency to precipitate.

It is well known that formulating a drug as a solid dispersion in a matrix can facilitate faster dissolution, increased apparent solubility and greater bioavailability. These terms are known to one of skill in the art. As defined by Chiou and Riegelman (“Pharmaceutical Applications of Solid Dispersion Systems”, Journal of Pharmaceutical Sciences, Vol. 60, No. 9, September 1971), the term solid dispersion refers to the “dispersion of one or more active ingredients in an inert carrier or matrix at solid state prepared by the melting (fusion), solvent, or melting-solvent method.” When providing this definition, physical mixtures or “The dispersion of a drug or drugs in a solid diluent or diluents by traditional mechanical mixing is not include in this category.” This definition of solid dispersions is applicable to this disclosure, and describes a mixture of a drug and polymer such that the drug is partially or completely dissolved in the polymer. As used here, the term solid solution is a special case of a solid dispersion in which all of the drug in a drug-polymer mixture is molecularly dispersed or dissolved in the polymer.

As used here, the term apparent solubility refers to a measure of how much solute can be dissolved in a solvent from a formulation or during a dissolution, and may exceed the equilibrium solubility. However, if the apparent solubility exceeds the equilibrium solubility, there will be a tendency for the solute to precipitate bout of solution and return partially to an undissolved form until the dissolved concentration equals the equilibrium solubility. This concept is known to one of skill in the art, and is exemplified by so-called “spring and parachute” patterns.

Methods of improving dissolution by producing solid dispersions can generally be described as solvent-based methods or melt-mix methods. There are variations of each, and each has found use in commercial scale production of solid dispersions.

In a preferred embodiment, the solid formulation will comprise a drug as the substance that is mixed with or dispersed in a polymer carrier. However, it will be apparent to one of skill in the art that the instant invention is not limited to drugs and/or polymers, and may include any chemical dispersed in a carrier. Also, said chemical and/or carrier need not be pharmaceutically acceptable.

For many drugs with poor aqueous solubility, dissolution of the pure drug is slow. The dissolution can be improved in a number of ways. For instance, mixing the drug with a hydrophilic polymer such as PVP (polyvinylpyrrolidone) can improve the dissolution rate of the drug compared with that of the pure drug form. Optionally, physical mixtures of a drug with other excipients, such as surfactants or other hydrophilic excipients, can result in improved dissolution compared to the pure crystalline drug form.

Other methods include particle size reduction, as is known in the art for micronization and forming nanoparticles. Still others include formation of co-crystals and chemical modifications such as salt formation.

Another approach to improving dissolution of a drug is to form solid solutions or solid dispersions. A solid solution is a molecular dispersion of the drug in a polymer or other carrier. A solid dispersion is a dispersion of drug particles in a polymer or other carrier, and often coexists with a solid solution. (A solid solution is a special case of a solid dispersion.)

When producing a solid dispersion using solvent-based methods, the drug and polymer are typically dissolved in a common solvent and uniformly mixed, and the solvent is then removed. Variations can be used in which the drug is dissolved in the solvent but the polymer is not dissolved, but may be solvated. When producing a solid dispersion using melt-mix methods, the polymer is melted or heated to a temperature in which it can be made to flow, and the drug is mixed into the molten polymer. Optionally, the drug can be melted and mixed with the melted polymer, then the mixture is allowed to cool.

Examples of this method and variations include solvent evaporation, co-precipitation, spray drying, lyophilization (freeze drying), kneading, imbibing of drug solutions into excipient pores, and gel entrapment. In most cases, the drug and carrier or polymer are both dissolved in a common solvent and mixed while in solution, but for some, the drug is dissolved in a solvent and the solution is introduced into the carrier matrix, followed by removal of the solvent by or other means. For drugs with poor aqueous solubility, organic solvents are typically employed because they can dissolve sufficient quantities of the drug, while water and polar solvents are not used because of their inability to dissolve sufficient quantities of the drug to allow mixing of the drug and carrier polymer in solution. Suitable organic solvents include, but not limited to, methanol, ethanol, propanol, ethylene chloride, methylene chloride, chloroform, and others. In addition, other nonpolar solvents can also be used.

The solvent-based methods are described below.

Solvent evaporation/co-precipitation. A drug and polymer are typically dissolved in a common organic solvent and mixed. The solvent is then evaporated to produce a solid dispersion.

Spray drying. A solid dispersion is formed by dissolving a drug and polymer in a suitable solvent, then the solution is sprayed through nozzle to form an aerosol in an evaporation chamber. The solvent evaporates leaving a solid dispersion in the form of a powder.

Lyophilization (freeze drying). A drug and carrier such as a polymer are dissolved in a common solvent, which is then frozen. The frozen solvent is then sublimed to obtain a lyophilized molecular dispersion.

Kneading. Physical mixtures of a drug and polymer are prepared, then small volumes of ethanol-water or similar solutions are added with mixing to produce a paste, which is then dried in an oven or vacuum.

Gel entrapment. A gel is formed by dissolving a polymer in an organic solvent, in which a drug is dissolved before or during dissolution of the polymer. After mixing, the solvent is evaporated using under vacuum, which results in solid dispersions.

Solution imbibing into pores. A method for depositing small drug particles in the pores of carriers is disclosed by Bellantone (US Patent Application, Publication US 2009/0130212, 2009). In this method, a drug is dissolved in an organic solvent, and the solution is imbibed into pores of a solid matrix. The solvent is chosen to allow dissolving as much drug as possible, and the matrix is chosen to be insoluble in the drug solution, thus preserving the pore structure. After imbibing, the solvent is removed by vacuum or other means of evaporation, causing the drug to precipitate and leaving small drug particles deposited in the pores of the matrix.

A variation of the gel entrapment method is disclosed by Buxton and Feldman (Solid Shaped Articles, U.S. Pat. No. 4,754,597, 1998.) In this method, hydrated gelatin is poured into molds and then the water is removed to leave a dry gelatin matrix. A solution of the drug in an organic solvent is dropped onto the top of the gelatin and allowed to imbibe, and the solvent is then, leaving the drug dispersed in the gelatin matrix.

Anti-solvent method. A drug and carrier are dissolved and mixed in an organic solvent to form a drug-carrier solution. The solution is then fed with supercritical carbon dioxide through a nozzle, resulting in precipitation, then the supercritical fluid and organic solvent are rapidly removed, leaving solid dispersion particles.

Eutectic mixtures. A drug and second crystalline solid are dissolved in a common solvent in a particular ratio, then the solvent is removed. The ratio is chosen so the drug occupies sites in the crystal lattice or interstitial spaces to form a eutectic mixture. This can result in a solid solution, which may be present as crystalline or amorphous particles. This may also be produced by melting both the drug and crystalline solid, mixing then cooling.

Examples of the melt-mix and variations are described below and include:

Melting method. In this method, a physical mixture of a drug and carrier such as a polymer is made. The mixture is then heated to above the melting point of all the components and optionally mixing of the molten mixture may be done. The molten mixture is then allowed to cool to form a uniform solid. A variation of the melting method is the dropping method, in which a physical mixture of drug and polymer is melted and subsequently dropped or poured onto a cool plate, inducing solidification

Hot melt extrusion (HME). Solid dispersions can be prepared by forcing a polymer through an extruder at a temperature above the glass transition of the polymer, and optionally above the melting temperature of the drug or polymer. The polymer may not be completely melted, but is heated sufficiently so it has flow properties that allow it to be extruded. In the extruder, fine powders or granules of the drug are added and mixed with the molten polymer by the extruder process. After extrusion, the mixture is allowed to cool and further processed.

Melt agglomeration. Solid dispersions can be prepared either by heating the carrier and drug above the melting temperature of the carrier, or by spraying a dispersion of drug in molten carrier onto a heated excipient by using a high shear mixer.

In addition to producing solid dispersions by solvent-based and melt-mix type methods, other methods have been used to produce solid formulations with improved dissolution. For instance, small particles of a drug can be formed in a polymer or carrier by mechanical mixing. An example of this method is co-grinding, in which a physical mixture of drug and carrier is mixed in a blender, then the mixture is further subjected to milling with small steel spheres to reduce the particle size of the drug powder or granules. In this method, there is a mechanically induced heating, which can result in forming amorphous regions of the drug.

Another alternative method is to anneal the drug and polymer mixture at elevated temperatures. In this method, a physical mixture of drug and polymer is allowed to stand at elevated temperatures for extended periods of time (referred to in this context as annealing), then allowed to cool to room temperature. While standing at the elevated temperature, drug molecules can migrate and disperse as individual molecules by diffusing into the polymer. If allowed to go on for a long enough time, the drug will eventually become completely dissolved in the polymer, provided the amount of drug does not exceed what can dissolve in the polymer at said elevated temperature. If the amount of drug present exceeds the drug-in-polymer solubility, the drug molecules migrate and disperse in the polymer until the concentration of drug molecules in the polymer becomes uniform and equal to the solubility at the elevated temperature, with the excess drug remaining present as drug particles.

Each method offers advantages and disadvantages. In the solvent-based methods, the solvent used must be able to dissolve the drug, and preferably both the drug and the polymer. Since solid dispersions are typically formed for drugs with poor water solubility, an organic solvent is typically employed to dissolve the drug. In addition, the solvent must be in a liquid form and used in a sufficient quantity to dissolve the drug and the polymer, so a relatively large amount of solvent is needed compared to the amounts of the drug and polymer. Because the solvent may not be physiologically compatible, or because of regulatory requirements place limits on the amount of residual solvent left in the formulation after removal, complete removal of the solvent is important. In addition, environmental concerns arise because the evaporated solvent must be captured properly after removal from the formulation, and cannot be simply emptied into the general environment.

Melt-mix and annealing methods avoid the issues of solvent removal encountered with solvent-based methods, but introduce a different set of problems. For melt-mix methods, exposing a drug and polymer to elevated temperatures can lead to thermal breakdown of either or both. Further, the polymer must be heated to a temperature that is high enough so the flow properties allow the drug to be uniformly mixed in the molten polymer. This limits the choice of polymers, since not all polymers can achieve proper flow properties before reaching temperatures at which they physically or chemically break down, or at temperatures that are feasible in practice.

Annealing methods have the further disadvantage that they are typically slow, with many drug-polymer combinations requiring days to a week or more before the drug and polymer have equilibrated in the sense that the drug is molecularly uniformly distributed in the polymer, and no additional drug can molecularly disperse, either because all of the available drug has dispersed, or because the total amount of drug exceeds what can be dissolved in the given amount of polymer. In addition, high temperatures are required for extended periods, which can lead to thermal degradation during annealing for some drugs and polymers.

From the above discussion, it is apparent that it is desirable find a method of producing solid formulations with improved dissolution properties that can minimize the quantity of solvent used and minimize exposure to high temperatures.

The present invention discloses a method to produce solid formulations providing such improved dissolution by annealing a drug and polymer while in contact with an adjuvant, which will be referred to as annealing with an adjuvant. As used here, the term adjuvant refers to an agent that is in contact with mixtures of a drug and polymer during annealing that allow the annealing to be done at lower temperatures and/or for shorter times than required for annealing without the adjuvant. The quantity of adjuvant used is such that the all or nearly of the drug and polymer are undissolved in the adjuvant, and the polymer is not heated to a temperature at which it will melt. However, the polymer and/or drug may be plasticized by the adjuvant, thus increasing the ability of the drug to mix with the polymer by diffusion mechanisms. Annealing with an adjuvant may be done with or without mechanical mixing, for a time sufficient to allow the at least some of the drug to diffuse into the polymer. The adjuvant can be optionally removed, either during or after the annealing.

In a preferred embodiment, the adjuvant is a liquid solvent presented to a mixture of a solid drug and solid polymer mixture before or during annealing, for instance by drops onto the surface of the mixture. The amount of the adjuvant with which the annealing mixture is in contact is sufficient to cause plasticization of the polymer and/or drug, but is small enough so nearly none of the drug and polymer present in the mixture dissolve in the adjuvant before or during annealing. Annealing is typically done in an oven, and may optionally be done under a vacuum for all of part of the annealing time.

In another preferred embodiment, the adjuvant is a solvent in the vapor state, to which a mixture of a solid drug and solid polymer is exposed during annealing. Annealing is done in an oven and the adjuvant occupies the space in the oven while it also interacts with the mixture, and may optionally be done under a vacuum for all or part of the annealing time. The adjuvant may optionally be introduced into vapor phase in the oven after producing a vacuum. As used in this disclosure, the term vacuum refers to a reduced total pressure, and may also be referred to as a partial vacuum.

In other embodiments, the adjuvant may be a solid at room temperature but a melt at the annealing temperature.

The adjuvant may be an organic solvent or other nonpolar solvent, or it may be water or other polar solvent. It may also be a surfactant, including but not limited to surfactants such as TPGS (Vitamin E derivative), or plasticizers such as triacetin, dibutyl sebacate, propylene glycol, and others known in the art. The purpose of the adjuvant is not to act as a solvent for the drug or polymer, or to alter the chemical properties of the drug and polymer mixture, but instead to allow mixing of the drug and polymer to occur by annealing more readily. This can be due to plasticization of the drug or polymer, but is not limited to this mechanism.

In addition, the instant invention can be employed with mechanical agitation. For instance, mixtures of the drug and polymer can be granulated or mixed continuously or intermittently while exposed to an adjuvant vapor. Also, either the drug or polymer can be exposed to or annealed under the adjuvant liquid or vapor before being mixed with the other component(s) while exposed to the adjuvant liquid or vapor. In addition, the annealing process can be interrupted to switch adjuvants in the vapor phase or add additional liquid adjuvant, preferably followed by additional annealing. Also, if more than one drug is to be mixed with one or more polymer, the components can be mixed at the beginning of the process, or a subset can be mixed then annealed, followed by adding an additional component followed by annealing, etc. If exposed to liquid or vapor adjuvant in multiple steps, the same or different adjuvants can be employed at various stages of the process. Other possible variations would be apparent to one of skill in the art and could be selected based on routine experimentation.

Surprisingly, it has been found that annealing with an adjuvant produces solid formulations with improved dissolution without dissolving the drug and polymer, and without melting the polymer (or the polymer and drug).

It has also been surprisingly found that the annealing with an adjuvant produces solid formulations with improved dissolution by annealing at temperatures that are lower and/or require less time to anneal than required by annealing without an adjuvant.

It has further been surprisingly found that mechanical mixing of the components is not necessary during annealing with an adjuvant. In addition, it has been surprisingly found that the adjuvant does not require any specific manner of addition to or mixing with the drug and polymer mixture, and it can be added directly to the mixture (for instance, be dropped onto the mixture surface) or in a vapor state.

Further, it has surprisingly been found that the adjuvant may be organic or nonpolar, but it may also be water or aqueous, even when producing formulations comprising drugs that are poorly soluble in water.

It would be apparent to one of skill in the art that factors such as the choice of adjuvant, the temperature at which annealing is done, the duration of annealing, etc. could be optimized with routine optimization and experimentation.

It would also be understood by one of ordinary skill in the art that the solid drug-polymer mixtures can be subjected to further processing as part of producing a dosage form or formulation. Such processing is known in the art and includes, but is not limited to, techniques such as crushing, pulverized, granulation, sieving, mixing with excipients, compression into tablets, etc.

The instant invention has various advantages over other methods. For instance, solvent-based methods that involve dissolving the drug and possibly the polymer require using much more solvent than the amounts of adjuvant required by the instant invention. In turn, this leads to requiring removal of the solvent, which is wasteful and requires recovery of the solvent for environmental regulations. The instant invention uses adjuvants in much smaller amounts, thus significantly reducing the amount of residual adjuvant that must be removed.

Melt and mix methods present another set of difficulties associated with elevated temperatures, which can lead to chemical instabilities in many drugs and polymers, as well as drug polymer combinations. These are even more problematic in the case of annealing without an adjuvant, which must be done at elevated temperatures and for long periods of time, thus exposing the components of the mixture to extended periods of higher chemical degradation risk. The instant invention allows annealing to be done at lower temperatures and over shorter duration, thus minimizing the risk of chemical breakdown.

Also, for commercially applicable production scales, solvent-based methods and melt-mix methods both require complicated and expensive instruments. For instance, commercial scale batches are produced using solvent removal methods by spray drying, but spray drying equipment is expensive and requires extensive ongoing maintenance associated with cleaning nozzles, etc. In addition, it is cumbersome to use one spray dryer for more than one formulation. Similar considerations apply for hot melt extrusion (HME), which is the preferred method of producing commercial sized batches based on the melt-mix prototype. Again, HME uses expensive equipment that requires extensive ongoing maintenance, and is not well suited for producing more than one formulation per instrument. The instant invention avoids most of the problems associated with spray drying and HME, since the instant invention can work using simple vacuum ovens. These are inexpensive, and easy to clean and maintain. In addition, they are amenable to a wide range of batch sizes with similar instrumentation with little waste, ranging from as low as one gram to commercial scale.

### SUMMARY OF THE INVENTION

The present invention comprises a method to produce solid formulations of drug and polymer mixtures providing improved dissolution of the drug, by annealing a drug and polymer mixture while in contact with an adjuvant. This method will be referred to as annealing with an adjuvant. As used here, the term adjuvant refers to an agent that is in contact with mixtures of a drug and polymer during annealing, and that allow the annealing to be done at lower temperatures and/or for shorter times than required for annealing without the adjuvant. The amount of the adjuvant is such that nearly all of the drug and polymer are undissolved in the adjuvant, and the during annealing with an adjuvant the polymer is not heated to a temperature at which it will melt.

In this context, improved dissolution may be faster dissolution of the drug, increased extent of dissolution of the drug, or both. Annealing with an adjuvant is done to at least partially dissolve the drug in the polymer, and may produce solid dispersions of a drug in a polymer at least partly in the form of a solid solution, but is not limited to this mechanism of improving dissolution. In the following, the terms drug and polymer will be used to describe a preferred embodiment, but the concept refers to any substance with at least some solubility in a polymer or other carrier matrix.

The method comprises providing a mixture of a drug and solid polymer in any physical form, including a physical mixture of drug and polymer, and allowing the drug to migrate into the polymer by diffusion while the mixture is kept at an elevated temperature. The method further comprises adding an adjuvant to said physical mixture before or during the annealing.

The method still further comprises allowing the mixture of drug, polymer and adjuvant to stand at temperatures that may be elevated for time long enough for the drug to substantially equilibrate with the polymer and the solvent to be substantially completely removed from the mixture, most preferably by evaporation of the solvent.

In a preferred embodiment, the adjuvant is a liquid solvent presented to a mixture of a solid drug and solid polymer mixture before or during annealing, for instance by drops onto the surface of the mixture. The amount of the adjuvant with which the annealing mixture is in contact is sufficient to cause plasticization of the polymer and/or drug, but is small enough so nearly all of the drug and polymer present in the mixture are not dissolved in the adjuvant before or during annealing. Annealing is done in an oven, and may optionally be done under a vacuum for all of part of the annealing time.

In another preferred embodiment, the adjuvant is a solvent in the vapor state, to which a mixture of a solid drug and solid polymer is exposed during annealing. Annealing is done in an oven while the adjuvant occupies the space in the oven and also interacts with the mixture, and may optionally be done under a vacuum for all of part of the annealing time. The adjuvant may optionally be introduced into vapor phase in the oven after producing a vacuum or partial vacuum.

In other embodiments, the adjuvant may be a solid at room temperature but a melt at the annealing temperature.

The adjuvant may be an organic or other nonpolar liquid solvent, or it may be water or other polar solvent. It may also be a surfactant, including but not limited to surfactants such as TPGS (Vitamin E derivative), or various polysorbate nonionic surfactants such as Tween-20, Tween-40 and Tween 80, among others. The adjuvant may also be a plasticizer such as triacetin, dibutyl sebacate, propylene glycol, and others known in the art. The purpose of the adjuvant is not to act as a solvent for the drug or polymer, or to alter the chemical properties of the drug and polymer mixture, but instead to allow mixing of the drug and polymer to occur by annealing more readily. This can be due to plasticization of the drug or polymer, but is not limited to this mechanism.

In its broadest sense, the invention relates to producing solid mixtures of any molecule and any solid carrier in which said molecule is at least partially soluble or miscible by annealing with an adjuvant. While the example describes binary mixtures of one drug (as the dissolving substance) plus one polymer (as the carrier or matrix), the example and concepts can be extended to include systems comprised of other dissolving substances and mixtures of three or more components.

In addition, the instant invention applies to mixing more than one drug or molecule in a carrier that may be a single polymer, a mixture of polymers, or a mixture of one or more polymers plus excipients. Suitable excipients include, but are not limited to, ionic and neutral surfactants, and plasticizers. Also, the resulting mixture of said drug in said polymer can be further processed using techniques known in the art to produce pharmaceutical formulations for administration by various routes to the body, including oral, injectable, transdermal, sublingual, buccal, intranasal, and inhalation routes.

In essence, the invention provides a method for producing a formulation in which the drug is in a more readily dissolvable form, or the carrier is in a form that facilitates faster and/or more complete dissolution of the drug, or both, compared to the pure drug or the drug in a physical mixture. This may be accomplished by employing annealing with an adjuvant to produce a solid dispersion of a drug or other small molecule in a polymer. However, the instant invention is not limited to this mechanism for improved dissolution.

Using a drug as an example of a small dissolving molecule, the invention provides a method comprising:
a) providing a drug and polymer in a known ratio ranging from 0.1 to 99% drug w/w, preferably 1 to 50%, even more preferably 2-30% drug;
b) mixing the drug and polymer, preferably as a physical mixture, and more preferably as a physical mixture in which the particle size has been reduced, for instance by milling or trituration;
c) adding an adjuvant to the physical mixture, or allowing the adjuvant to come into contact with the physical mixture, in an adjuvant amount such that the nearly all of the drug and polymer are undissolved in the adjuvant, preferably in a adjuvant weight that is less than the weight of the physical mixture;
d) placing the physical mixture plus adjuvant in a controlled environment at a temperature that is above room temperature and below the melting temperature of the drug or polymer, more preferably below the melting temperature of both the drug and the polymer, and still more preferably below the nominal glass transition temperature of the polymer in the absence of the adjuvant;
e) allowing the physical mixture plus adjuvant to anneal at said temperature for a period of time that is long enough for the drug to at least partially dissolve in the polymer by diffusion;
f) optionally removing the adjuvant from said mix of drug and polymer after;
g) allowing the annealed mixture of drug and polymer to cool.

In essence, the instant invention allows a substance such as a drug to dissolve in the polymer using less adjuvant than would be needed to dissolve all of the drug or all of the polymer, or both, followed by annealing at lower temperatures and for shorter times than those required for annealing without the adjuvant. It is thought that this is accomplished by using the small amount of the adjuvant to plasticize the polymer and/or drug, and allow them to molecularly mix at least partially by diffusion, which occurs faster due to the plasticization and higher temperature.

In this context, the term anneal refers to allowing a mixture to stand for a specified time at a temperature that is above room temperature (nominally 20° C.), then allowing the mixture to cool to room temperature. At the increased temperature, the drug and polymer mix by diffusion of drug molecules into the polymer, which occurs more rapidly because the drug and polymer both have more molecular kinetic energy compared to lower temperatures, thus increasing the mobility of the drug in the polymer as well as the ability of the drug molecules to separate from each other and enter the polymer by diffusion.

Diffusion of the drug molecule is due to migration of the drug molecules into the polymer by random thermal motion. The rate at which the drug diffuses into the polymer is influenced by the molecular motion of the drug and or polymer. For instance, if the molecular kinetic energy of the drug is increased, the molecules will have a tendency to spread out and diffuse into the polymer more rapidly, and display a higher diffusion coefficient. This is temperature sensitive, with higher temperatures resulting in more molecular kinetic energy and faster diffusion of the drug into the polymer.

A similar consideration applies to molecular motion of the polymer. More molecular motion of the polymer results in less resistance to diffusion of the drug into the polymer, and also might provide less steric hindrance to diffusion of the drug because of larger average separation of polymer molecules and more average void space or holes into and through which drug molecules can diffuse. This increased polymer molecular motion can be brought about by increasing the temperature, and is particularly pronounced above the polymer glass transition temperature Tg of the polymer during the annealing. It is known that the introduction of certain solvents or other small molecules can also result in increased molecular motion and lower the Tg of a polymer compared to its value in the absence of said solvents or small molecules. This is referred to as plasticization, and the solvent or other small molecules are referred to as plasticizers. Only small quantities of a solvent are needed to effectively plasticize a polymer, and are typically much less than the amount of solvent required to dissolve a substantial fraction of the polymer or the drug present in a mixture. This plasticization is an intended primary function of the adjuvant.

Thus, it is thought that adding small amounts of adjuvant to the physical mixture will increase the mobility of the polymer and drug molecules without increasing the temperature, or with a small increase in temperature, thus plasticizing the polymer and/or drug. This occurs because the adjuvant diffuses into the polymer and drug mixture, and interacts on a molecular scale to increase their molecular motion. Thus, the presumed role of the adjuvant is not to dissolve the drug or polymer in their entirety, but instead to interact with the mixture as a sort of plasticizer. Since plasticization requires much less adjuvant than dissolution of the mixture components, much smaller amounts of adjuvant are needed in annealing with an adjuvant than would be required in solvent-based methods such as coprecipitation, etc.

The plasticization effect of the adjuvant allows the polymer strands and dissolving substance molecules to increase their molecular motion, which lowers energetic barriers to diffusion. Thus, at any given temperature, mixing of the drug and polymer by molecular diffusion of the dissolving substance can occur more rapidly in the presence of the adjuvant than would occur in annealing without the adjuvant. This, in turn, allows producing solid dispersions and solid solutions by annealing with an adjuvant that requires lower temperatures and less time than would be required using annealing without the adjuvant.

Notably, plasticization of a polymer is not solely brought about by liquid adjuvants. It is known that dissolving excipients such as surfactants and pharmaceutically known plasticizers in a polymer also plasticize the polymer. Also, dissolving certain drugs in a polymer may also cause plasticization. Thus, while the adjuvant and/or other excipients may be the initial plasticizer that increases dissolution rates, the presence of the drug in the polymer can also increase the migration rate of the drug into the polymer, even after the adjuvant has been removed.

Importantly, because the adjuvant spreads out in the mixture by diffusion as part of introducing the plasticizer effect, it is not necessary to introduce the adjuvant in any particular manner. Thus, it can be introduced as drops in the mixture of the polymer and dissolving substance, or it can be introduced from the vapor phase directly. Further, the adjuvant can be mechanically mixed with polymer and substance mixture using methods such as blending or trituration, but this is not necessary.

It has unexpectedly been found that adding small amounts of an adjuvant before or during annealing with an adjuvant can produce solid mixtures with improved dissolution compared to the pure drug or physical mixtures of the drug and polymer.

It has also been unexpectedly found that the annealing with an adjuvant can be carried out effectively at temperatures below the nominal glass transition temperature Tg of the polymer in the absence of plasticization.

It has further been unexpectedly found that drug and polymer physical mixtures annealed with an adjuvant can produce solid solution formulations using shorter annealing times and lower annealing temperatures than would be required for annealing without said adjuvants.

It has been surprisingly found that said adjuvant is effective for this purpose without the need to dissolve the drug and polymer, and without the need to melt the polymer and drug. In some cases, the fraction of drug and/or polymer dissolved by the solvent is much lower than one percent. This is surprising because solvent-based methods require dissolving the drug and preferably the polymer as well, then removing the solvent, while melt-mix methods require melting the polymer.

It has further been surprisingly found that the adjuvant can be added to the drug-polymer mixture as a liquid before or during annealing with an adjuvant, or as a vapor during annealing. This is surprising because it is typically not possible to dissolve a drug or polymer in a adjuvant from the vapor phase.

If has still further been surprisingly found that the adjuvant can be water, water vapor, an aqueous solution, or any combination, even for drugs with poor aqueous solubility. This is particularly surprising because the conventional knowledge in the art teaches that be that water cannot be used to solubilize or otherwise change the dissolution properties of a drug with poor aqueous solubility in a solid formulation. Specifically, it is surprising that using small amounts of water that cannot dissolve any substantial fraction of the drug is an effective adjuvant to produce a mixture with improved dissolution.

It has also been unexpectedly found that annealing drug and polymer physical mixtures with an adjuvant can produce solid solutions using shorter annealing times and lower annealing temperatures than would be required for annealing without said adjuvants.

In one such embodiment, the drug and polymer mixture to be annealed is a physical mixture. However, it can be a mixture of any form of the drug and polymer, including but not limited to, crystalline or amorphous forms, already formed drug-polymer solid dispersions containing drug particles, drug-polymer solid dispersions plus additional drug, and the like.

In one embodiment, the adjuvant is added to the drug and polymer mixture in a liquid form that is simply dropped onto the drug and polymer mixture. The adjuvant may be a pure solvent or a solution containing one or more solutes. In another embodiment, the adjuvant is not added in a liquid form, but instead would be added in a vapor form by annealing the drug and polymer mixture in the presence of an adjuvant vapor, such as ethanol vapor or water vapor. In this embodiment, the annealing could be done in the presence of a vapor phase for a period of time, followed by removal of the adjuvant vapor with subsequent annealing in an environment void of the adjuvant vapor, during which annealing and adjuvant that had accumulated in the drug and polymer mixture from the vapor phase is then removed. Also, the drug and polymer mixture can be continuously or intermittently blended while annealing in the presence of the adjuvant vapor.

Numerous variations of the method are possible, such as annealing with an adjuvant while mixing in a tumbler or simply annealing with an adjuvant in stationary vials or enclosures. Also, the temperature can be varied during the annealing, additional adjuvant can be added during annealing, the vapor phase can be changed during annealing. In addition, if the adjuvant is added from the vapor phase, the vapor pressure can be varied. These are examples, but are not meant to limit the scope of embodiments of the instant invention.

The plasticization in the instant invention is preferably accomplished using water or ethanol, but is not limited to those substances. Other adjuvant substances in the liquid or vapor phase can include organic solvents or any volatile liquid. In addition, other agents that act as plasticizers can be used in small amounts in the drug-polymer mixture, including but not limited to surfactants such as ionic and nonionic surfactants, and plasticizers known in the art.

Suitable polymers include, but are not limited to, the group comprising various grades of: PVP (polyvinylpyrrolidone, povidone), PVP-VA (vinylpyrrolidone/vinyl acetate copolymer or copovidone), crospovidone, hydroxypropylmethylcellulose, hydroxypropylmethylcellulose phthalate, hydroxypropylmethylcellulose acetate succinate, hydroxypropylcellulose, hydroxypropylcellulose acetate phthalate, polyethylene oxide, gelatin, carbomer, cellulose acetate phthalate, cellulose acetate terephthalate, cellulose acetate isophthalate, carbomer, starch glycolate, carboxymethylcellulose, croscarmellose, methylcellulose, methylcellulose acetate phthalate, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate, and Soluplus®.

In a preferred embodiment, the instant invention is directed towards improving the dissolution of drugs with poor solubility and/or dissolution in water and aqueous based solutions. However, other drugs or compounds, even those with good solubility and/or dissolution in water and aqueous based solutions, are within the scope of the instant invention. The drugs that can be formulated using the instant invention include many pharmacological classes of compounds, but the instant invention is particularly directed toward drugs with poor solubility in water, such as BCS II and IV compounds.

The percent of said drug in the mixture can range from about less than one percent to greater than 99%. In a practical embodiment, it is preferable for the mixture to contain the drug in a weight percent (weight of drug to weight of the mixture) from 1 to 50% drug, and more preferably from 2 to 30% drug. The mixture can be binary and contain only the drug and polymer, or contain more than one drug, more than one polymer, additional excipients including those know to act as plasticizers, and mixtures thereof.

Also, the time required for the annealing with an adjuvant varies with the drug and polymer, but can be determined by routine optimization for each set of components

The annealed mixture of the instant invention may be further formulated into pharmaceutical dosage forms comprising a therapeutically effective amount of the mixture. Although pharmaceutical dosage forms for oral administration, such as tablets and capsules, are envisaged, the composition of the present invention can also be used to prepare other pharmaceutical dosage forms, such as for rectal, vaginal, ocular or buccal administration, or the like.

It will also be apparent to those skilled in the art that these dosage forms may also include pharmaceutically useful excipients such as disintegrants, diluents, fillers, lubricants, glidants, colorants and flavors.

Suitable disintegrants might include, but not be limited to, crosslinked polymers such as crospovidone (crosslinked polyvinylpyrrolidone), croscarmellose (crosslinked sodium carboxymethylcellulose), and sodium starch glycolate. Suitable diluents or fillers might include, but not be limited to, sucrose, dextrose, mannitol, sorbitol, starch, micro-crystalline cellulose, and others known in the art, and mixtures thereof. Lubricants and glidants may also be used in certain dosage forms, and are typically used when producing tablets. Suitable lubricants and glidants may include, but not be limited to, magnesium stearate, stearic acid, sodium lauryl sulfate, magnesium lauryl sulfate, colloidal silica, talc, hydrogenated vegetable oils, and mixtures thereof. Coloring agents may also be added in accordance with the present invention, and may include but not be limited to titanium dioxide and dyes suitable for food. Flavors may be chosen from synthetic flavor oils and aromatics or natural oils, extracts from plants, leaves, flowers, fruits, etc. mixtures thereof. These may include, but not be limited to, oils such as cinnamon, wintergreen, peppermint, spearmint, bay, anise, eucalyptus, thyme. Other flavors may include vanilla, citrus oils such as lemon, orange, grape, lime and grapefruit, and fruit essences such as apple, banana, pear, peach, strawberry, raspberry, cherry, plum, pineapple, apricot, etc.

### Definitions

As used herein, the following terms are described. In most cases, the intended meaning is consistent with the usual meaning as understood by one of skill in the art. However, they are defined or described below for completeness or definiteness within the context of the disclosure of the instant invention.

Physical mixtures comprise one or more drug(s), one or more carrier(s), and optionally excipients in any form by simple mechanical mixing such that the drug and polymer are not molecularly dispersed in each other. In the context of the disclosure of the instant invention, it will refer to a mixture of at least one solid, typically with another solid or semi-solid, and most typically will refer to a mixture resulting in a solid form of two or more materials such that the process does not substantially result in a molecular dispersion, such as mechanical mixing by mortar and pestle or dry granulation.

The term drug refers to any chemical, approved for marketing or not, that is intended for medicinal use. However, drugs refer to a preferred embodiment of the instant invention, and the term drug is used in the descriptions and examples. The instant invention is not limited to drugs and may include other chemicals or agents without a medicinal or pharmacologically beneficial or toxic activity.

The term anneal refers to subjecting a material or mixture of materials to an elevated temperature that is typically above room temperature (approximately 20-22° C.), for a period of time, then allowing said material or mixture of materials to cool to room temperature.

The term adjuvant refers to a chemical or agent that can increase the rate of mixing during annealing at a given temperature, or be used to lower the temperature at which mixing by annealing occurs. An adjuvant may be in the form of a liquid or vapor at the annealing temperature, and may be an organic solvent, water, or an aqueous solution. It may also be a solid at room temperature but a liquid at the annealing temperature.

A formulation comprises a material or mixture of materials that has optionally been processed by technique of the instant invention and/or techniques known in the art, and which may optionally include excipients, to produce a final form of a material or mixture with optionally intended properties or characteristics. In the context of the disclosure of the instant invention, the formulation is typically in a solid form.

Organic solvent refers to non-aqueous, carbon based solvents such as methanol, ethanol, propanol, ethylene chloride, methylene chloride, chloroform, and the like. Most typically, these solvents are in the liquid state at room temperature, but they may be in a liquid and/or gaseous form at elevated temperatures used for annealing.

Carrier refers to any material in which a drug or solute, or multiple drugs and solutes, is to be dispersed. In the context of the disclosure of the instant invention, it is typically a solid polymer, but it optionally may be a non-polymeric material.

Solute refers to the substance that is intended to be dissolve in a solvent or carrier.

Solvent refers to the material in which a solute is dissolved, and may be a liquid, semi-solid or solid. In the context of the disclosure of the instant invention, it is typically a liquid.

A solution is a molecular dispersion of a solute in a solvent.

Dissolved refers to a mixture of a solute in a solvent, in which solute molecules are substantially separated from each other and molecularly dispersed in the solvent. The solvent can be a liquid or solid. In turn, the term undissolved refers to the physical state of the solute material in which the solute molecules are not molecularly dispersed in the solvent, but instead are aggregated to primarily interact with themselves. The undissolved form of a solute is most typically a solid, but may also be a liquid or gas.

Dissolution is the process of removing solute molecules from a physical arrangement or state in which they are undissolved to one in which they are dissolved in a solvent.

Solubility refers to the equilibrium or thermodynamic solubility, which is the highest concentration of a solute, such as a drug or other agent, that can be stably dissolved in a solvent, so that there is no thermodynamic tendency for the dissolved solute to crystallize or precipitate out of the solution and into an undissolved state.

Aqueous solubility is the maximum amount of a solute that can be dissolved in water, or an aqueous solution such as a buffer solution.

The glass transition temperature, or Tg, refers to the nominal temperature at which a polymer or other substance undergoes the glass transition in the absence of plasticizers.

Oral dosage form refers to a dosage form or formulation intended to be administered by the oral route. The drug dissolution from an oral dosage form occurs in the fluids in the GI tract, typically referring to the stomach and/or intestines and/or colon.

Absorption refers to the process by which drug molecules pass from one side of a physiological membrane to the other. Typically, absorption is to the side of a membrane or barrier that is in contact with the bloodstream, or liquids that lead to contact with the bloodstream.

Bioavailability refers to the fraction of a dose that is given that is absorbed and becomes available to the systemic circulation.

### DESCRIPTION OF THE FIGURES

FIG. 1 shows dissolved concentration vs time data obtained from dissolution of pure indomethacin (circles) and physical mixtures of indomethacin plus PVP (polyvinylpyrrolidone), with indomethacin weight percents of 10% (squares) and 20% (triangles), as described in Example 1.

FIG. 2 shows dissolved concentration vs time data obtained from dissolution of annealed mixtures of indomethacin plus PVP annealed under a vacuum for 24 hours at 100° C. in the absence of an adjuvant. The squares represent dissolution data from mixtures containing 10% indomethacin, and the triangles represent dissolution data from mixtures containing 20% indomethacin (by weight), as described in Example 2.

FIG. 3 shows dissolved concentration vs time data obtained from dissolution of annealed mixtures of indomethacin plus PVP annealed under a reduced pressure for 24 hours at 100° C. in the presence of ethanol vapor as the adjuvant. The squares represent dissolution data from mixtures containing 10% indomethacin, and the triangles represent dissolution data from mixtures containing 20% indomethacin (by weight), as described in Example 3.

FIG. 4 shows dissolved concentration vs time data obtained from dissolution of annealed mixtures of indomethacin plus PVP annealed under a reduced pressure for 24 hours at 100° C. in the presence of water vapor as the adjuvant. The squares represent dissolution data from mixtures containing 10% indomethacin, and the triangles represent dissolution data from mixtures containing 20% indomethacin (by weight), as described in Example 4.

FIG. 5 shows dissolved concentration vs time data obtained from dissolution of annealed mixtures of indomethacin plus PVP hydrated with liquid water as the adjuvant, annealed under a reduced pressure for 24 hours at 100° C. The squares represent dissolution data from mixtures containing 10% indomethacin, and the triangles represent dissolution data from mixtures containing 20% indomethacin (by weight), as described in Example 5.

### EXAMPLES

The examples below show data resulting from a small set of experiments. The examples are meant for illustration only, and are not to be considered as limiting the scope of materials that can be used, including the drug, polymer and adjuvant, and choice of a polymer and/or adjuvant for a given drug. In addition, they should not be considered to limit the experimental parameters, such as the temperature, amount of adjuvant used, whether the adjuvant is introduced as a liquid or vapor, the time for annealing, or the magnitude of the vacuum (if any). All of these factors can be satisfactorily determined with routine optimization by one of skill in the art. In addition, it is possible to determine by routine optimization when the process is over, to optimize the manufacturing process.

### Example 1

Binary physical mixtures of the drug indomethacin and the polymer PVP (polyvinylpyrrolidone K29/32), both used as received, were mixed using a mortar and pestle for 15 minutes, then approximately 6.25 grams were transferred to a glass plate. The mixtures contained indomethacin percents of 10% and 20% by weight (one part indomethacin to nine parts PVP, and one part indomethacin to four parts PVP by weight). Each physical mixture was placed in a vacuum oven of approximately 1.5 liter volume. A vacuum was induced over approximately one minute to a total pressure of one inch Hg or lower, then the valves were closed and no further vacuum was induced. The oven temperature was then set to 40° C., and the mixture was allowed to stand for 2 hours under the vacuum.

Dissolution experiments were done using a USP Type II (paddle type) apparatus, dissolving 150 mg of formulation in 500 mL of water buffered to a pH of 2.5 using phosphate buffer at 37° C. This corresponded to 15 mg of indomethacin per 500 mL of dissolution medium for the 10% indomethacin formulation, and 30 mg of indomethacin per 500 mL of dissolution medium for the 20% indomethacin formulation.

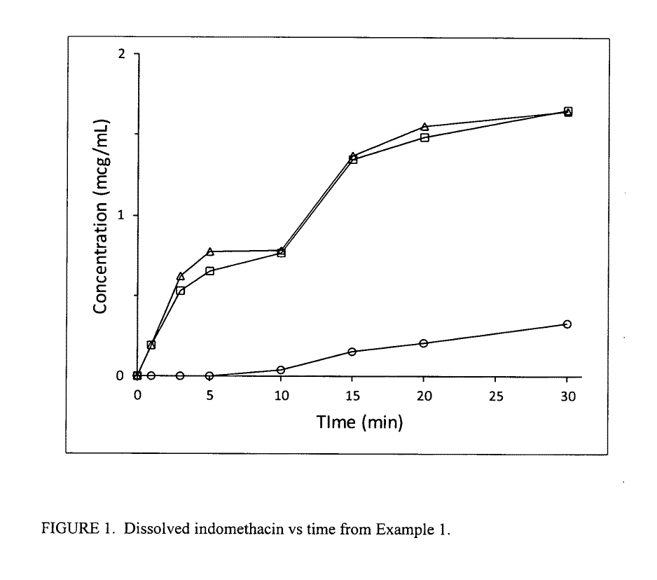

In addition, pure indomethacin was dissolved in a similar USP type II apparatus for 30 minutes for comparison. The results are shown in FIG. 1.

### Example 2

Binary physical mixtures of the drug indomethacin and the polymer PVP (polyvinylpyrrolidone K29/32), both used as received, were mixed using a mortar and pestle for 15 minutes, then approximately 6.25 grams were transferred to a glass plate. The mixtures contained drug percents of 10% and 20% by weight. Each physical mixture was placed in a vacuum oven of approximately 1.5 liter volume, and a vacuum was induced over approximately one minute to a total pressure of one inch Hg or lower, then the valves were closed and no further vacuum was induced. The oven temperature was then set to 100° C., and the mixture was allowed to anneal for 24 hours in the oven.

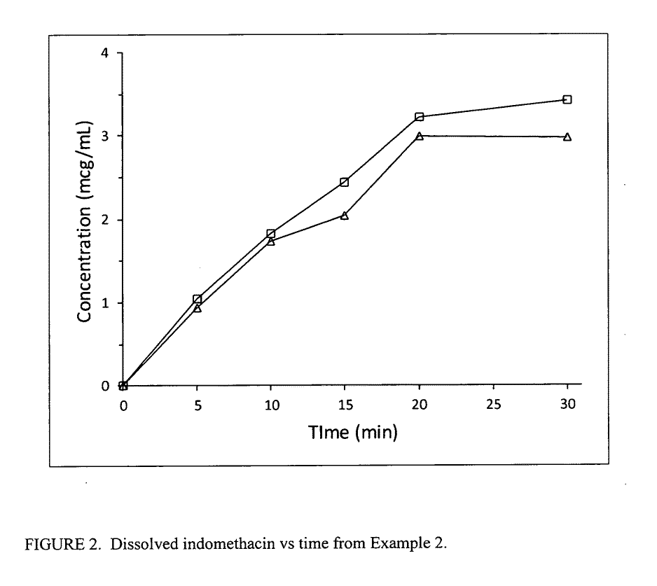

Dissolution experiments were done using a USP Type II (paddle type) apparatus, dissolving 150 mg of formulation in 500 mL of water buffered to a pH of 2.5 using phosphate buffer at 37° C. This corresponded to 15 mg of indomethacin per 500 mL of dissolution medium for the 10% indomethacin formulation, and 30 mg of indomethacin per 500 mL of dissolution medium for the 20% indomethacin formulation. The results are shown in FIG. 2.

### Example 3

Binary physical mixtures of the drug indomethacin and the polymer PVP (polyvinylpyrrolidone K29/32), both used as received, were mixed using a mortar and pestle for 15 minutes, then approximately 6.25 grams were transferred to a glass plate. The mixtures contained drug percents of 10% and 20% by weight. Each physical mixture was placed in a vacuum oven of approximately 1.5 liter volume along with a beaker containing 8 mL of ethanol (approximately 6.3 grams). A vacuum was induced over approximately one minute to a total pressure of one inch Hg or lower, then the valves were closed and no further vacuum was induced. The oven temperature was then set to 100° C., and all of the ethanol evaporated. The mixture was allowed to anneal for 24 hours in the oven in the presence of the ethanol vapor as the adjuvant.

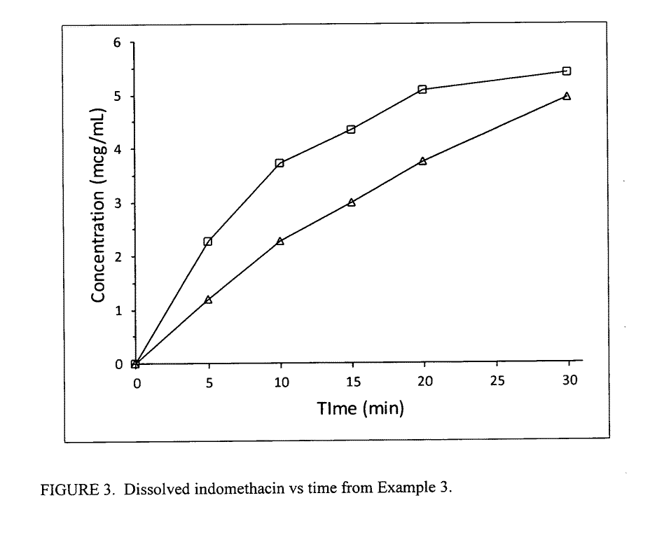

Dissolution experiments were done using a USP Type II (paddle type) apparatus, dissolving 150 mg of formulation in 500 mL of water buffered to a pH of 2.5 using phosphate buffer at 37° C. This corresponded to 15 mg of indomethacin per 500 mL of dissolution medium for the 10% indomethacin formulation, and 30 mg of indomethacin per 500 mL of dissolution medium for the 20% indomethacin formulation. The results are shown in FIG. 3.

### Example 4

Binary physical mixtures of the drug indomethacin and the polymer PVP (polyvinylpyrrolidone K29/32), both used as received, were mixed using a mortar and pestle for 15 minutes, then approximately 6.25 grams were transferred to a glass plate. The mixtures contained drug percents of 10% and 20% by weight. Each physical mixture was placed in a vacuum oven of approximately 1.5 liter volume along with a beaker containing 6 mL of water. A vacuum was induced over approximately one minute to a total pressure of one inch Hg or lower, then the valves were closed and no further vacuum was induced. The oven temperature was then set to 100° C., and all of the water evaporated. The mixture was allowed to anneal for 24 hours in the oven in the presence of the water vapor as the adjuvant.

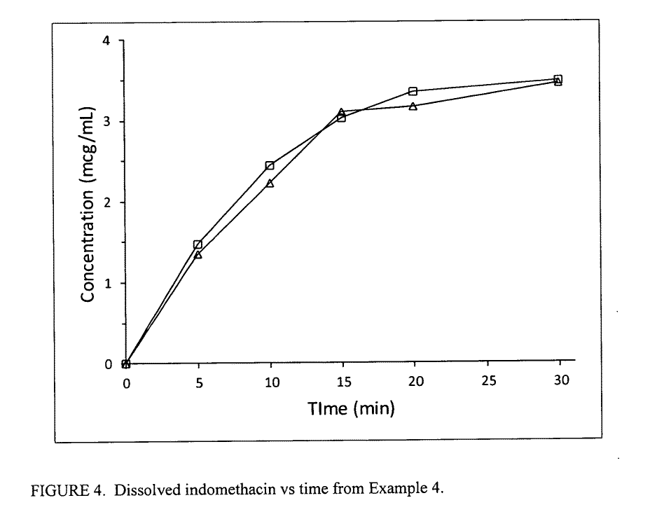

Dissolution experiments were done using a USP Type II (paddle type) apparatus, dissolving 150 mg of formulation in 500 mL of water buffered to a pH of 2.5 using phosphate buffer at 37° C. This corresponded to 15 mg of indomethacin per 500 mL of dissolution medium for the 10% indomethacin formulation, and 30 mg of indomethacin per 500 mL of dissolution medium for the 20% indomethacin formulation. The results are shown in FIG. 4.

### Example 5

Binary physical mixtures of the drug indomethacin and the polymer PVP (polyvinylpyrrolidone K29/32), both used as received, were mixed using a mortar and pestle for 15 minutes, then approximately 6.25 grams were transferred to a glass beaker or petri dish. The mixtures contained drug percents of 10% and 20% by weight. Subsequently, 6 mL of water was added to the indomethacin plus PVP physical mixture and mixed for 15 minutes to form a gel. The gel was then placed in a vacuum oven of approximately 1.5 liter volume, and a vacuum was induced over approximately one minute to a total pressure of one inch Hg or lower, then the valves were closed and no further vacuum was induced. The oven temperature was then set to 100° C., and the mixture was allowed to anneal for 24 hours in the oven.

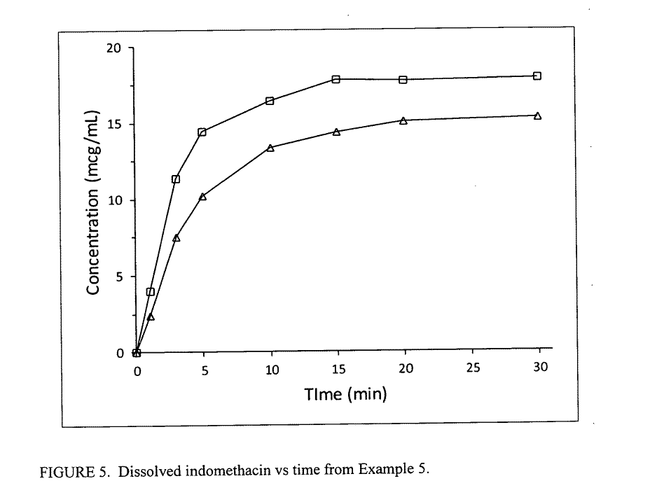

Dissolution experiments were done using a USP Type II (paddle type) apparatus, dissolving 150 mg of formulation in 500 mL of water buffered to a pH of 2.5 using phosphate buffer at 37° C. This corresponded to 15 mg of indomethacin per 500 mL of dissolution medium for the 10% indomethacin formulation, and 30 mg of indomethacin per 500 mL of dissolution medium for the 20% indomethacin formulation. The results are shown in FIG. 5.

### Example 6

Binary physical mixtures of the drug indomethacin and the polymer PVP (polyvinylpyrrolidone K29/32), both used as received, were mixed in various ratios, so the drug weight fraction ranged from 2.5-30% w/w (compared to the total physical mixture). Mixing was done for 15 minutes using a mortar and pestle. Solid solutions were then prepared by annealing and annealing with an adjuvant.

Annealing was done by transferring weighed samples of the physical mixtures into glass vials, which were then stored in an oven without inducing a vacuum at 130° C. for 6 days to equilibrate and allow the indomethacin to distribute and dissolve in the PVP. After annealing, the samples were allowed to cool to room temperature.

Annealing with an adjuvant was done by transferring weighed amounts of the physical mixtures into glass vials, then adding the adjuvant ethanol dropwise to the surface of the physical mixture in each vial in an adjuvant weight equal to approximately 25% of each dry drug-polymer physical mixture sample. No mixing of the adjuvant and physical mixture was done after adding the ethanol drops. The drug, polymer and adjuvant were then allowed to anneal in an oven at 70° C., with some samples annealing for 4 hours, some for 8 hours and some for 12 hours. After annealing with an adjuvant for each specified time, the samples were allowed to cool to room temperature.

Differential scanning calorimetry was performed on samples of all formulations produced by annealing with and without an adjuvant. This was done by heating the annealed mixtures from room temperature to 180° C., which is approximately 15-20° C. above the melting temperature of pure indomethacin. The absence of melting endotherms in a DSC endotherm was taken to mean that all of the drug in the mixture was dissolved. For the samples produced by annealing without an adjuvant, it was seen that all of the drug dissolved up to a concentration of approximately 15% by weight when annealed for 6 days at 130° C. On the other hand, it was seen that all of the indomethacin was dissolved in the PVP up to about the same 15% by weight concentration after annealing with an adjuvant was done for 12 hours at 70° C. In addition, the DSC did not show any evidence of ethanol for the formulations produced using annealing with an adjuvant, indicating that the small amount of adjuvant was completely or nearly completely removed from the mixture during the annealing.

### Discussion

In addition to Examples 1-6, other experiments were done in which the solubility of indomethacin in aqueous buffer at pH 2.5 was determined. This was done by placing an excess amount of indomethacin solid (approximately 100 mg) in 20 mL of the buffer and stirring at a constant temperature of 37° C. for three days, after which an aliquot was taken and filtered through a Watman Anotop-10 20 nm filter, then assayed by HPLC for the dissolved indomethacin concentration in the solution. Subsequently, the temperature of the remaining indomethacin-buffer mixture was raised to approximately 95° C. and kept at that temperature with stirring for an additional 24 hours. After 24 hours, the dissolved indomethacin concentration was again determined by taking an aliquot, filtering through a 20 nm Anotop-10 filter, and assaying by HPLC. (Thus, for this determination, the dissolved concentration reflected three days at 37° C. then an additional day at 95° C.) It was found that the dissolved indomethacin concentration at 37° C. after three days was less than 1 mcg/mL, and it was approximately 27 mcg/mL after stirring for three days at 37° C. plus an additional day at 95° C.

FIG. 1 shows the results from Example 1. The pure indomethacin dissolution in the aqueous buffer at 37° C. was much slower than indomethacin in physical mixtures with PVP, which is a hydrophilic polymer and presumably improved the indomethacin dissolution by facilitating wetting of the indomethacin by the aqueous dissolution medium. After 30 minutes, however, even the dissolution from the physical mixtures result in a dissolved indomethacin concentration of only approximately 1.6 to 1.7 mcg/mL. These values are consistent, even if not equal to, the solubility determined independently of below one mcg/mL. (It should be noted that for drugs with very low solubility, variations in the dissolved concentrations that are fractionally significant can be small in terms of the actual concentration values.) Thus, for the purposes of comparison, an estimate for the solubility of indomethacin in the aqueous buffer at 37° C. can be taken as approximately one to two mcg/mL.

FIG. 2 shows the effects of annealing without the presence of a adjuvant liquid or vapor, as described in Example 2. The dissolution of the indomethacin from both mixtures (10% and 20% indomethacin) drug was improved compared to the pure indomethacin and physical mixtures of indomethacin and PVP, being faster and to a greater extent over the first 30 minutes. The dissolved concentration at 30 minutes was approximately 3.4 mcg/mL for dissolution from the 10% indomethacin mixture and approximately 3.0 mcg/mL for the 20% indomethacin mixture, which were slightly higher than the estimated solubility from the solubility experiments and Example 1.

FIG. 3 shows the effects of annealing in the presence of ethanol vapor, as described in Example 3. The dissolution of the indomethacin from both mixtures (10% and 20% indomethacin) drug was further improved over the results from physical mixtures and annealed mixtures in the absence of ethanol. The dissolved concentration at 30 minutes was approximately 5.4 mcg/mL for dissolution from the 10% indomethacin mixture and approximately 4.9 mcg/mL for the 20% indomethacin mixture, both of which are significantly greater than the estimated solubility value of from the solubility experiments and Example 1.

FIG. 4 shows the effects of annealing in the presence of water vapor, as described in Example 4. The dissolution of the indomethacin from both mixtures (10% and 20% indomethacin) drug was improved over the results from physical mixtures and pure indomethacin of Example 1, and similar to those of the mixtures that were annealed in Example 2, with dissolved concentrations at 30 minutes of approximately 3.5 mcg/mL for dissolution from both the 10% and 20% indomethacin mixtures. Thus, although indomethacin is poorly soluble in water, using water vapor as an adjuvant during the annealing did not hinder producing a mixture with improved dissolution compared to the pure indomethacin and physical mixtures with no annealing.

FIG. 5 shows the effects of annealing in the presence of water vapor, as described in Example 5. In this example, liquid water was added to the physical mixture to form a gel, which was then annealed. The dissolution of the indomethacin from both mixtures (10% and 20% indomethacin) drug was vastly improved over the results from physical mixtures and pure indomethacin of Example 1, as well as the annealed mixtures from Examples 2-4, with dissolved concentrations at 30 minutes of approximately 17.9 mcg/mL for dissolution from the 10% indomethacin mixture and 15.3 mcg/mL for dissolution from the 20% indomethacin mixture. However, by the same arguments given in the preceding discussion of example 4 results, the indomethacin was substantially not dissolved in the water at any point in the process of Example 5.

The results observed from Example 4 and especially Example 5 are surprising. Both disclose methods of processing a mixture containing indomethacin using water as the adjuvant, resulting in a mixture that provides improved dissolution in an aqueous medium. Given the very poor solubility of indomethacin in water, this result is surprising and not expected based on prior art teachings.

Specifically, the total quantity of indomethacin annealed in Examples 4 and 5 was approximately 62 mg (65,000 mcg) for the 10% indomethacin mixture, and approximately 125 mg (125,000 mcg) for the 20% indomethacin mixture. Using a solubility value 2 mcg/mL for indomethacin in water at 37° C., 6 mL of water would only dissolve 12 mcg of indomethacin, which is negligible compared to 65,000 and 125,000 mcg present in the annealed mixtures. Even at 95° C. (just below the annealing temperature), the solubility of indomethacin in water was approximately 27 mcg/mL, so 6 mL of water would only dissolve 162 mcg of indomethacin, representing approximately 0.26% of the indomethacin present in the 10% mixture that was annealed with the adjuvant, and approximately 0.13% of the indomethacin present in 20% indomethacin mixture. In addition, it is likely that even less water was available to dissolve indomethacin because some was in the vapor phase during annealing at 100° C., and some was associated with the PVP due to its strongly hydrophilic nature, thus reducing the amount of water available to dissolve the indomethacin. These calculations and considerations indicate that very nearly all of the indomethacin was not dissolved in the adjuvant during the annealing process.

Example 6 shows that the annealing in the presence of ethanol produces solid solutions when the indomethacin weight fraction does not exceed approximately 15%, and solid dispersions when it is above 15%. This is because annealed mixtures with drug loads above 15% would be expected to produce a saturated solid solution corresponding to 15% by weight of indomethacin dissolved in PVP, with the rest of the indomethacin being undissolved as particles of indomethacin or mostly indomethacin in the mixture.

In all of the examples, annealing with and without adjuvants was done below the nominal glass transition temperature of the PVP. The nominal Tg of the PVP grade used in these examples is approximately 165-170° C. in the absence of any plasticization by solvents or adjuvants. However, it is appreciated by those of skill in the art that the presence of small amounts of water or ethanol in PVP, even at levels far below what would be required to dissolve the PVP, results in lowering of the Tg below the nominal value. Mixing of the indomethacin and PVP occurred at the temperatures in the examples. However, Example 6 shows that the addition of ethanol as the adjuvant led to faster mixing at lower temperatures (70° C. over 12 hours with the adjuvant versus 130° C. over 6 days with no adjuvant).

I claim:

1. A method for making a mixture of a substance and polymer that produces improved dissolution compared to a pure substance or a physical mixture of said substance and said polymer, comprising
a) providing a mixture of a polymer and a substance that can at least partially dissolve in the polymer;
b) adding an adjuvant to said polymer and said substance mixture in an amount such that said substance and said polymer are nearly completely undissolved in the adjuvant during the process;
c) placing said adjuvant plus said polymer and said substance mixture in a controlled environment with an elevated temperature that is higher than room temperature but lower than the melting temperature of said polymer in the presence of said adjuvant;
d) allowing said adjuvant plus said polymer and said substance mixture to anneal for a sufficient time in the controlled temperature for said substance to at least partially dissolve in said polymer.

2. A method of claim 1 wherein said substance is a drug.

3. A method of claim 1 wherein said adjuvant is added in a liquid form to said mixture of substance and polymer.

4. A method of claim 1 wherein said adjuvant is added in a vapor form to said mixture of substance and polymer.

5. A method of claim 1 wherein said substance and polymer are mixed as powders to form a physical mixture.

6. A method of claim 1 wherein less than 10% of said drug in the mixture is dissolved in the adjuvant.

7. A method of claim 1 wherein less than 1% of the drug in the mixture is dissolved in the adjuvant.

8. A method of claim 1 wherein less than 10% of the polymer is dissolved in the adjuvant.

9. A method of claim 1 wherein less than 1% of the polymer is dissolved in the adjuvant.

10. A method of claim 1 wherein said elevated temperature is lower than the nominal glass transition temperature of said polymer in the absence of plasticizers.

11. A method of claim 1 wherein said elevated temperature is lower than the melting temperature of said substance.

12. A method of claim 1 wherein said adjuvant is added during annealing.

13. A method of claim 1 wherein said adjuvant is removed during or after annealing.

14. A method of claim 1 wherein said substance is in its crystalline form.

15. A method of claim 1 wherein said substance is in a polymorphic form.

16. A method of claim 1 wherein said substance is in an amorphous form.

17. A method of claim 1 wherein said adjuvant is an organic solvent.

18. A method of claim 1 wherein said adjuvant is water or an aqueous solution.

19. A method of claim 1 wherein said annealing is done under a partial vacuum.

20. A method of claim 1 wherein said adjuvant is removed during or after annealing.

21. A method for making a solid dispersion of a substance dispersed in a polymer, comprising
a) providing a mixture of a polymer and a substance that can at least partially dissolve in the polymer;
b) adding an adjuvant to said polymer and said substance mixture in an amount such that the substance and polymer are nearly completely undissolved in the adjuvant during the process;
c) placing the adjuvant plus said polymer and substance mixture in a controlled environment with an elevated temperature that is higher than room temperature but lower than the nominal glass transition temperature of said polymer;
d) allowing the adjuvant plus said polymer and substance mixture to anneal for a sufficient time in the controlled temperature for the substance to dissolve in and equilibrate with the polymer.

22. A method of claim 21 wherein said substance is a drug.

23. A method of claim 21 wherein said adjuvant is added in a liquid form to said mixture of substance and polymer.

24. A method of claim 21 wherein said adjuvant in the vapor form is put in contact with said mixture of substance and polymer.

25. A method of claim 21 wherein said substance and polymer are mixed as powders to form a physical mixture.

26. A method of claim 21 wherein less than 10% of the drug in the mixture is dissolved in the adjuvant.

27. A method of claim 21 wherein less than 1% of the drug in the mixture is dissolved in the adjuvant.

28. A method of claim 21 wherein less than 10% of the polymer is dissolved in the adjuvant.

29. A method of claim 21 wherein less than 1% of the polymer is dissolved in the adjuvant.

30. A method of claim 21 wherein said elevated temperature is lower than the nominal glass transition temperature of said polymer in the absence of plasticizers.

31. A method of claim 21 wherein said elevated temperature is lower than the melting temperature of said substance.

32. A method of claim 21 wherein said adjuvant is added during annealing.

33. A method of claim 21 wherein said adjuvant is removed during or after annealing.

34. A method of claim 21 wherein said substance is in its crystalline form.

35. A method of claim 21 wherein said substance is in a polymorphic form.

36. A method of claim 21 wherein said substance is in an amorphous form.

37. A method of claim 21 wherein said adjuvant is an organic solvent.

38. A method of claim 21 wherein said adjuvant is water or an aqueous solution.

39. A method of claim 21 wherein said annealing is done under a partial vacuum.

40. A method of claim 21 wherein said adjuvant is removed during or after annealing.
